(19) United States
(12) Patent Application Publication
(10) Pub. No.: US 2026/0209272 A1
(43) Pub. Date: Jul. 23, 2026

(54) BIOPROTECTANT PEPTIDES FOR THE PREVENTION OF LATE BLIGHT

(71) Applicant: Innatrix Inc., Durham, NC (US)

(72) Inventors: Theodore W. KAHN, Apex, NC (US); Yuanhan WU, Durham, NC (US); Jiarui LI, Cary, NC (US); Sohaila DAR, Durham, NC (US); John SALMERON, Durham, NC (US)

(21) Appl. No.: 19/141,272

(22) PCT Filed: Dec. 20, 2023
(86) PCT No.: PCT/US2023/085246

§ 371 Date: Jun. 19, 2025

Related U.S. Application Data
(60) Provisional application No. 63/476,196, filed on Dec. 20, 2022.

(51) Int. Cl. C07K 7/06; A01N 37/46; A01P 3/00

(52) U.S. Cl. CPC C07K 7/06; A01N 37/46; A01P 3/00

(57) ABSTRACT

This invention relates to the field of molecular biology. Provided are novel amino acid sequences that encode bioprotectant proteins and peptides. These proteins and peptides and the amino acid sequences that encode them are useful in preparing bioprotectant formulations. Herein provided are identified polypeptides that inhibit oomycete infection of plants. The polypeptides are protein ligands binding to oomycete cellulose synthases thereby inhibiting or ameliorating infection by oomycetes.

### CROSS REFERENCE TO RELATED APPLICATIONS

This application claims priority from U.S. Provisional Application No. 63/476,196 filed on Dec. 22, 2022, hereby incorporated by reference in its entirety.

### SEQUENCE LISTING

The Sequence Listing associated with this application is provided in text format in lieu of paper copy, and is hereby incorporated by reference into the specification. The name of the text file containing the Sequence Listing is “97974-403929_Sequence Listing_ST26.” The xml file is 3.58 KB, was created on Dec. 19, 2023, and is being submitted electronically via Patent Center, concurrent with the filing of this specification.

### FIELD OF THE INVENTION

This invention relates to the field of molecular biology. Provided are novel amino acid sequences that encode anti-oomycete and pesticidal proteins and peptides. These proteins and peptides and the amino acid sequences that encode them are useful in preparing antimicrobial, anti-oomycete, and pesticidal formulations.

### BACKGROUND

The potato is the world's fourth most important food crop and by far the most important vegetable. Potatoes are currently grown commercially in nearly every state of the United States. Annual potato production exceeds 18 million tons in the United States and 300 million tons worldwide. The popularity of the potato derives mainly from its versatility and nutritional value. Potatoes can be used fresh, frozen or dried, or can be processed into flour, starch or alcohol. They contain complex carbohydrates and are rich in calcium, niacin and vitamin C.

Late blight causes $6.7 billion in annual losses for potato production globally. The traditional management of late blight depends heavily on preventative fungicides that may also protect against oomycetes, such as metalaxyl fungicide, mancozeb, cymoxanil, and dimethomorph. The fungicides only provide partial protection, and resistance presents an ever-rising challenge to their efficacy. New chemical fungicides such as mandipropamid are being used to prevent late blight outbreaks. However, it takes at least 10 years and more than $300 million to develop one product.

Furthermore, the efficiency of these treatments, and in general of phytosanitary treatments, is very low; it is estimated that less than 0.1% of the active ingredient applied to crops is actually able to impact the targeted pathogen. The remainder accumulates in the soil and from there, by washout of the contaminated grounds, can reach and pollute surface water and underground aquifers, becoming a hazard for freshwater organisms and human beings. Fungicides accumulating in soil can damage arthropods, earthworms, fungi, bacteria, protozoa and in general all the organisms that contribute to the function and structure of soil. The chronic exposure of phytosanitary products to useful insects, like bees, and to wild birds can cause reductions in their reproductive capacity, with consequences at the level of species and ecosystems, while exposure to high concentrations can even cause the death of individuals. Pets can also be influenced by exposure to phytosanitary products. Some active ingredients have poor degradability and hence remain in the environment for a long time. For example, organochlorine insecticides, such as DDT, were identified in the surface water of the United States 20 years after their use had been forbidden. Moreover, the phytosanitary products that enter the food chain can undergo the phenomenon of biomagnification. This term means the tendency of some chemical substances to become ever more concentrated the farther up one rises in the trophic chains. Consequently, the concentrations accumulating in the tissues of organisms can be many times higher than in the surrounding environment.

One approach to inhibiting plant-pathogenic fungal and oomycete activity has been to identify and isolate peptides, polypeptides, and proteins exhibiting activity against plant pathogenic fungi (Bowles, 1990; Brears et al., 1994). Examples of anti-oomycete and antifungal peptides, polypeptides, and proteins include chitinases, cysteine-rich chitin-binding proteins, β-1,3-glucanases, permatins (including zeamatins), thionins, ribosome-inactivating proteins, and non-specific lipid transfer proteins, and are believed to play important roles in natural plant defense against fungal infection. The potential use of these proteins to control pathogens in transgenic plants has been reported, for example, in European Patent Application 0 392 225.

### SUMMARY OF THE INVENTION

Herein provided are polypeptide protein ligands identified to bind to oomycete cellulose synthase and that are efficient at treating and preventing late blight and cucurbit downy mildew.

The polypeptide NoPv1 has been previously disclosed.

NoPv1: RLTAQCRL

The polypeptides of the present disclosure are:

Peptide V1: RITAQIRL
Peptide V2: RNTAQIP

The present disclosure is related to amino acid sequences that are either (i) not found anywhere in nature or (ii) not found in nature as applied to the organism of interest. According to some embodiments, any peptide sequence having less than 100% identity to a reference sequence shall differ from any naturally occurring amino acid sequence of the same size by at least one amino acid (e.g., by substitution, deletion, or insertion).

The present disclosure, in part, relates to the fact that the amino acid sequences provided may be chemically synthesized into polypeptides, as is well known. The present disclosure relates, in some embodiments, to a bacterial cell comprising an expression vector. For example, a bacterial cell, such as E. coli, may comprise an expression vector comprising, in a 5′ to 3′ direction, (a) an expression control sequence; (b) an expressible nucleic acid sequence (e.g., a nucleic acid sequence encoding an exogenous polypeptide) operably linked to the expression control sequence; and (c) a 3′ termination sequence operably linked to the expressible nucleic acid sequence. A bacterial cell may comprise, for example, an expression vector comprising, in a 5′ to 3′ direction, (a) an expression control sequence; (b) an exogenous nucleic acid sequence operably linked to the expression control sequence; and/or (c) a 3′ termination sequence operably linked to the exogenous nucleic acid sequence, wherein the exogenous nucleic acid sequence comprises a nucleic acid sequence encoding a polypeptide with at least about 95% identity to a polypeptide sequence selected from the group consisting of RLTAQIRL, and RNTAQIPL.

The present disclosure relates, in some embodiments, to an isolated, purified anti-oomycete protein or peptide, comprising an amino acid sequence having at least 90% sequence identity to an amino acid sequence selected from the group consisting of amino acid sequences RLTAQIRL, and RNTAQIPL.

The present disclosure relates, in other embodiments, to the isolated, purified anti-oomycete protein or peptide having the sequence RLTAQIRL, or RNTAQIPL, further comprising a plant apoplast, vacuolar, or endoplasmic reticulum targeting amino acid sequence at its N-terminus. In some preferred embodiments, the protein or peptide is fused with a protein that increases expression or solubility, such as, but not limited to thioredoxin. The isolated, purified nucleotide sequence can be reverse translated from RLTAQIRL, and RNTAQIPL, and codon-optimized for expression in a plant of interest.

The plant of interest may be a food, crop plant, ornamental plant, or tree. Food or crop plants include soybean, wheat, maize, sugarcane, rice, tomato, grape, or potato. Crops may include, but are not limited to lettuces, ornamentals, peppers, eggplants, tomatoes, onion, citrus fruits, tobacco, cotton, potatoes, soy, legumes (e.g., alfalfa, clover, pea bean), or cucurbits (e.g., pumpkins, zucchini, cantaloupe, watermelon, cucumber).

The present disclosure relates, in some embodiments, to a transgenic plant, cells of which contain an anti-oomycete protein or peptide comprising an amino acid sequence having at least 70% sequence identity to an amino acid sequence selected from the group consisting of amino acid sequences RLTAQIRL, and RNTAQIPL. The anti-oomycete protein or peptide may, in other embodiments, further comprise a plant apoplast, vacuolar, or endoplasmic reticulum targeting amino acid sequence. In some embodiments, said anti-oomycete protein or peptide is present in said cells in an anti-oomycete effective amount.

In preferred embodiments, the anti-oomycete protein or peptide inhibits damage to said plant caused by a Phytophthora or other oomycete species. In other embodiments, the anti-oomycete protein or peptide inhibits infection or ameliorates damage to a plant by oomycetes. The oomycetes that are affected by the anti-oomycete protein or peptide may include: Phytophthora infestans, Hyaloperonospora arabidopsidis, Phytophthora ramorum, Phytophthora sojae, Phytophthora capsici, Plasmopara viticola, Phytophthora cinnamomi, Phytophthora parasitica, Pythium ultimum, Albugo candida, Bremia lactucae, Phytophthora nicotianae, or Pseudoperonospora cubensis, or any combination thereof.

In some embodiments, the peptide of sequence RLTAQIR or RNTAQIPL could be chemically synthesized, or could be made by fermentation in microorganisms such as E. coli, Pseudomonas fluorescens, yeast, or others.

In some embodiments, the fermented peptide might be purified before being sprayed, or the dead microorganism containing the peptide might be sprayed, or a crude culture supernatant might be sprayed.

In yet other embodiments, the peptide may be applied as a seed treatment or soil drenching, as is known by those skilled in the art.

In other embodiments, the peptide could be sprayed on transgenic plants that express a gene that protects them from pathogens, pests, or abiotic stress. The peptide could also be combined with an RNAi treatment against pathogens, pests, or abiotic stress.

In other embodiments, an anti-oomycete composition comprises an anti-oomycete protein or peptide comprising an amino acid sequence having at least 90% sequence identity to an amino acid sequence selected from the group consisting of amino acid sequences RLTAQIRL, and RNTAQIPL, and any combination thereof. In preferred embodiments, the anti-oomycete protein or peptide, or combination thereof, is present in an effective amount. In other embodiments, the anti-oomycete composition is present with an agriculturally or pharmaceutically acceptable carrier, diluent or excipient. In yet other embodiments, the anti-oomycete composition is present in an aquaculturally effective amount.

In some embodiments the anti-oomycete protein or peptide, or combination thereof is in present in a concentration in the range of from about 0.1 microgram per milliliter to about 500 milligrams per milliliter. In other embodiments, the anti-oomycete protein or peptide, or combination thereof, is present in a concentration in the range of from about 5 micrograms per milliliter to about 250 milligrams per milliliter. In some embodiments, the anti-oomycete composition has a pH in the range of from about 3 to about 10. Moreover, the agricultural composition may be formulated with one or more additives selected from the group consisting of an inert material, a surfactant, a stabilizer, and a solvent.

In some preferred embodiments, the anti-oomycete composition is formulated in a mixture of one or more other agrochemical active agents selected from the group consisting of a pesticidally active substance, a fertilizer, an insecticide, an attractant, a sterilizing agent, an acaricide, a nematicide, an herbicide, and a growth regulator. In some embodiments the pesticidally active substance is selected from the group consisting of a fungal antibiotic and a chemical fungicide. In embodiments the fungal antibiotic or chemical fungicide is selected from the group consisting of a polyoxine, a nikkomycin, a carboxyamide, an aromatic carbohydrate, a carboxine, a morpholine, a sterol biosynthesis inhibitor, and an organophosphate or any combination thereof. In other embodiments, the anti-oomycete composition may further comprise another agriculturally active ingredient such as a pesticide, herbicide, nematicide, or fertilizer. In some embodiments the anti-oomycete composition is effective against pathogens resistant to treatment by commercial antifungal products.

The anti-oomycete composition inhibits, deters, or prohibits the growth of a susceptible oomycete species. In some embodiments the anti-oomycete composition inhibits, deters or prohibits the growth of oomycetes including Phytophthora. In other embodiments, the anti-oomycete composition inhibits, deters, or prohibits the growth of Phytophthora infestans, Hyaloperonospora arabidopsidis, Phytophthora ramorum, Phytophthora sojae, Phytophthora capsici, Plasmopara viticola, Phytophthora cinnamomi, Phytophthora parasitica, Pythium ultimum, Albugo candida, Bremia lactucae, Phytophthora nicotianae, or Pseudoperonospora cubensis, or any combination thereof.

In some embodiments, a method of controlling, combating, or inhibiting a susceptible oomycete, comprises contacting said susceptible oomycete with a transgenic plant, cells of which comprise and express an amino acid sequence encoding an anti-oomycete protein or peptide comprising an amino acid sequence having at least 90% sequence identity to an amino acid sequence selected from the group consisting of amino acid sequences RLTAQIRL, and RNTAQIPL, and a plant apoplast, vacuolar, or endoplasmic reticulum targeting amino acid sequence at the N-terminus of said anti-oomycete protein or peptide.

A composition is provided for prevention of late blight. The composition comprises either: the polypeptide as described above or any combination thereof, and an agrochemical or a carrier; or any combination of the polypeptides.

### BRIEF DESCRIPTION OF THE DRAWINGS

The above and other aspects, features, and advantages of the present invention will be better understood from the following detailed descriptions taken in conjunction with the accompanying drawing(s), all of which are given by way of illustration only, and are not limitative of the present invention, in which:

FIG. 1: is a photograph of untreated detached potato leaves, 6 days after inoculation with Phytophthora infestans.

FIG. 2: is a photograph of NoPv1-treated detached potato leaves, 6 days after inoculation with Phytophthora infestans.

FIG. 3: is a photograph of Peptide V1-treated detached potato leaves, 6 days after inoculation with Phytophthora infestans.

FIG. 4: is a photograph 6 of Peptide V2-treated detached potato leaves, 6 days after inoculation with Phytophthora infestans.

FIG. 5: is a photograph of detached potato leaves treated with Bonide Mancozeb Flowable with Zinc, 6 days after inoculation with Phytophthera infestans.

FIG. 6: is a photograph of detached potato leaves treated with NoPv1, Peptide V1 and Peptide V2, without Phytophthora infestans inoculation.

FIG. 7: is a photograph of detached potato leaves from a Phytophthora-resistant variety and a Phytophthora-susceptible variety, with and without treatment with Peptide V1, 6 days after inoculation with Phytophthora infestans.

FIG. 8: is a photomicrograph of Phytophthora infestans zoospores with and without treatment with Peptide V1 that had been exposed to direct sunlight for 48 hours, demonstrating that the peptide remains active.

FIG. 9 is a graph demonstrating that Peptide V1 retains its activity in the presence of other agrochemical active ingredients.

FIG. 10 is a graph demonstrating disease severity in plants treated with various concentrations of Peptide V1 with and without LI-700.

FIG. 11 is a collection of photographs depicting efficacy of Peptide V1, chemically synthesized or fermented in E. coli.

FIG. 12 is a photograph of detached cucumber leaves from a Pseudoperonospora cubensis-susceptible variety, with and without treatment with Peptide V1 with LI700, 3 days after inoculation with Pseudoperonospora cubensis.

FIG. 13 is a graph of an E. coli expression vector containing Peptide V1 fused to the E. coli trxA gene (thioredoxin).

FIG. 14 are gels demonstrating purification of a Peptide V1 fusion protein expressed in E. coli.

FIG. 15 is a pictorial representation of the pathogen protein that is the target of Peptide V1, as described in Daras, G.; Templalexis, D.; Avgeri, F.; Tsitsekian, D.; Karamanou, K.; Rigas, S. Updating Insights into the Catalytic Domain Properties of Plant Cellulose synthase (CesA) and Cellulose synthase-like (Csl) Proteins. Molecules 2021, 26, 4335. https://doi.org/10.3390/molecules26144335.

### DETAILED DESCRIPTION

The present invention is drawn to compositions and methods for preventing or ameliorating the effects of pathogens.

This description is not intended to be a detailed catalog of all the different ways in which the invention may be implemented, or all the features that may be added to the invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 C.F.R. §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gin; Q), glutamic acid (Glu; E), glycine (Gly; G), histidine (His; H), isoleucine (lie; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). As used herein, an “X” or “Xaa” in an amino acid sequence denotes that the amino acid in that position can be any of the 20 known amino acid or can be any of the enumerated amino acids recited herein.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

As utilized in accordance with the present disclosure, the following terms, unless otherwise indicated, shall be understood to have the following meanings:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the word “and/or” refers to and encompasses any and all possible combinations of one or more of the associated listed items, as well as the lack of combinations when interpreted in the alternative, “or.”

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1° C., preferably ±0.5° C. Where the term “about” is used in the context of this invention (e.g., in combinations with temperature or molecular weight values) the exact value (i.e., without “about”) is preferred.

As used herein, phrases such as “between about X and Y”, “between about X and about Y”, “from X to Y” and “from about X to about Y” (and similar phrases) should be interpreted to include X and Y, unless the context indicates otherwise.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence-based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

“Activity” of the pesticidal proteins of the invention means that the pesticidal proteins function to prevent or ameliorate the infection of the pathogen. “Pesticidal” is defined as a toxic biological activity capable of controlling a pest or pathogen, such as an insect, nematode, fungus, bacteria, oomycete, or virus, either by killing or destroying them, or by blocking their ability to infect or damage a plant. A “pesticidal agent” is an agent that has pesticidal activity. An “insecticidal agent” is an agent that has insecticidal activity.

The phrases “bioprotectant peptide”, “bioprotectant protein”, “antifungal protein”, “anti-oomycete protein”, “anti-oomycete peptide”, or “antifungal peptide” as used herein refer to proteins and peptides that exhibit any one or more of the following characteristics: inhibiting or retarding the growth of fungal or oomycete cells; killing fungal or oomycete cells; disrupting or retarding stages of the fungal or oomycete life cycle, such as spore germination, sporulation, or mating; and/or disrupting fungal or oomycete cell infection, penetration, or spread within a plant. The net effect is thus to limit, decrease, or eliminate fungal or oomycete pathogenesis and/or damage to a plant.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

As used herein, a “codon optimized” sequence means a nucleotide sequence wherein the codons are chosen to reflect the particular codon bias that a host cell or organism may have. This is typically done in such a way to preserve the amino acid sequence of the polypeptide encoded by the nucleotide sequence to be optimized. In certain embodiments, the DNA sequence of the recombinant DNA construct includes a sequence that has been codon optimized for the cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant.

The terms “comprising,” “including,” and “having” are intended to be inclusive and mean that there may be additional elements other than the listed elements.

A “colorant” as used herein acts as a visual product identifier for product branding and application. Colorants can include, but are not limited to, dyes and pigments, inorganic pigments, organic pigments, polymeric colorants, and formulated pigment coating dispersions available in a variety of highly concentrated shades.

To “deliver” a composition or a toxic or effective protein means that the composition or toxic or effective protein comes in contact with a pest or pathogen, which results in a toxic or other effect and control of the pest or pathogen. The composition or toxic or effective protein can be delivered in many recognized ways, including but not limited to formulated protein composition(s), sprayable protein composition(s), transgenic crops, a bait matrix, or any other art-recognized protein delivery system.

The term “domain” refers to a set of amino acids conserved at specific positions along an alignment of sequences of evolutionarily related proteins. While amino acids at other positions can vary between homologs, amino acids that are highly conserved at specific positions indicate amino acids that are likely essential in the structure, stability or function of a protein. Identified by their high degree of conservation in aligned sequences of a family of protein homologs, they can be used as identifiers to determine if any polypeptide in question belongs to a previously identified polypeptide group.

“Exogenously” applied as used herein refers to an application to the outside of a plant surface. A plant surface can be any external plant surface, for example a cuticle, a trichome, a leaf, a root hair, seed coat, etc.

“-Associated” or “-like” polypeptides as used herein refers to polypeptides derived from or structurally similar to the recited polypeptide but having an amino acid sequence and/or source distinct from the recited polypeptide.

A “foliar treatment” as used herein refers to a composition that is applied to the above ground parts or foliage of a plant or plant part and may have leaves, stems, flowers, branches, or any aerial plant part, for example, scion.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

“Gene of interest” refers to any nucleic acid molecule which, when transferred to an organism, such as a bacteria or a plant, confers upon the bacteria or plant a desired trait such as antibiotic resistance, virus resistance, insect resistance, disease resistance, or resistance to other pests, herbicide tolerance, abiotic stress tolerance, male sterility, modified fatty acid metabolism, modified carbohydrate metabolism, improved nutritional value, improved performance in an industrial process or altered reproductive capability. The “gene of interest” may also be one that is transferred to bacteria or plants for the production of commercially valuable enzymes or metabolites in the plant.

“Injection” as described herein can be used interchangeably with vaccination or immunization and provides a process whereby the bioactive priming polypeptides are delivered

“Inoculation” means to intentionally try to infect a plant or detached plant part by adding a pathogen. A “plant” refers to but is not limited to a monocot plant, a dicot plant, or a gymnosperm plant. The term “plant” as used herein includes whole plants, plant organs, progeny of whole plants or plant organs, embryos, somatic embryos, embryo-like structures, protocorms, protocorm-like bodies, and suspensions of plant cells. Plant organs comprise, shoot vegetative organs/structures (e.g., leaves, stems and tubers), roots, flowers and floral organs/structures (e.g., bracts, sepals, petals, stamens, carpels, anthers and ovules), seed including embryo, endosperm, and seed coat and fruit (the mature ovary), plant tissue (e.g., phloem tissue, vascular tissue, ground tissue, and the like) and cells (e.g., guard cells, egg cells, trichomes and the like). The class of plants that can be used in the methods described herein is generally as broad as the class of higher plants, specifically angio-sperms monocotyledonous (monocots) and dicotyledonous (dicots) plants and gymnosperms. It includes plants of a variety of ploidy levels, including aneuploid, polyploid, diploid, haploid, homozygous and hemizygous. The plants described herein can be members of the Solanum genus.

The term “isolated” nucleic acid molecule, polynucleotide or protein is a nucleic acid molecule, polynucleotide or protein that no longer exists in its natural environment. An isolated nucleic acid molecule, polynucleotide or protein of the invention may exist in a purified form or may exist in a recombinant host such as in a transgenic bacterium or a transgenic plant. Therefore, a claim to an “isolated” nucleic acid molecule, as enumerated herein, encompasses a nucleic acid molecule when the nucleic acid molecule is comprised within a transgenic plant genome.

Late blight. A potato disease caused by the oomycete Phytophthora infestans and also known as ‘potato blight’ that can infect and destroy the leaves, stems, fruits, and tubers of potato plants, and can also infect and damage many other plant species.

A “nucleic acid molecule” or “nucleic acid sequence” is a segment of single- or double-stranded DNA or RNA that can be isolated from any source. In the context of the invention, the nucleic acid molecule is typically a segment of DNA. In some embodiments, the nucleic acid molecules of the invention are isolated nucleic acid molecules.

Operably linked. Combining two or more molecules in such a fashion that in combination they function properly in a plant cell. For instance, a promoter is operably linked to a structural gene when the promoter controls transcription of the structural gene

Plant. As used herein, the term “plant” includes but is not limited to angiosperms and gymnosperms such as potato, tomato, tobacco, alfalfa, lettuce, carrot, strawberry, sugarbeet, cassava, sweet potato, soybean, maize, turf grass, wheat, rice, barley, sorghum, oat, oak, eucalyptus, walnut, and palm. Thus, a plant may be a monocot or a dicot. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent. A “selected plant species” may be, but is not limited to, a species of any one of these “plants.”

Plant Parts. As used herein, the term “plant parts” (or a potato plant, or a part thereof) includes but is not limited to protoplast, leaf, stem, root, root tip, anther, pistil, seed, embryo, pollen, ovule, cotyledon, hypocotyl, flower, tuber, eye, tissue, petiole, cell, meristematic cell, and the like.

A “plant cell” as used herein refers to any plant cell and can comprise a cell at the plant surface or internal to the plant plasma membrane, for example, an epidermal cell, a trichome cell, a xylem cell, a phloem cell, a sieve tube element, or a companion cell.

A “polynucleotide” refers to a polymer composed of many nucleotide monomers covalently bonded in a chain. Such “polynucleotides” includes DNA, RNA, modified oligo nucleotides (e.g., oligonucleotides comprising bases that are not typical to biological RNA or DNA, such as 2′-0-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

A “polypeptide” as described herein refers to any protein, peptide, or polypeptide.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plants genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule incorporated into its genome. As a result of such genomic alteration, the recombinant plant is distinctly different from the related wild-type plant.

Regulatory sequences. Refers to those sequences which are standard and known to those in the art that may be included in the expression vectors to increase and/or maximize transcription of a gene of interest or translation of the resulting RNA in a plant system. These include, but are not limited to, promoters, peptide export signal sequences, introns, polyadenylation, and transcription termination sites. Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567: 81, 1993). In engineering a plant system to affect the rate of transcription of a protein, various factors known in the art, including regulatory sequences such as positively or negatively acting sequences, enhancers and silencers, as well as chromatin structure may have an impact. The present invention provides that at least one of these factors may be utilized in engineering plants to express a protein of interest. The regulatory sequences of the present invention are native genetic elements, i.e., are isolated from the selected plant species to be modified.

The terms “substitution,” “insertion” or “addition,” and “deletion” are used herein with reference to amino acid or nucleotide sequences. A “substitution” refers to a replacement of one or more nucleotides or amino acids by different nucleotides or amino acids, respectively. An “insertion” or “addition” is that change in a nucleotide or amino acid sequence which has resulted in the addition of one or more nucleotides or amino acid residues, respectively, as compared to the naturally occurring sequence. A “deletion” is defined as a change in either nucleotide or amino acid sequence in which one or more nucleotides or amino acid residues, respectively, are absent. Amino acid substitutions are typically of single residues; insertions usually will be on the order of from about 1 to 20 amino acids, although considerably larger insertions may be tolerated. Deletions range from about 1 to about 20 residues, although in some cases deletions may be much larger. Substitutions, deletions, insertions, or any combination thereof may be used to arrive at a final variant polypeptide. Generally, a few amino acids are changed to minimize the alteration of the molecule. However, larger changes may be tolerated in certain circumstances. In certain embodiments, amino acid substitutions can be the result of replacing one amino acid with another amino acid having similar structural and/or chemical properties, such as the replacement of an isoleucine with a valine, i.e., conservative amino acid replacements. Insertions or deletions may optionally be in the range of 1 to 5 amino acids. In embodiments, substitutions can be made in accordance with known “conservative substitutions.” A “conservative substitution” refers to the substitution of an amino acid in one class by an amino acid in the same class, where a class is defined by common physicochemical amino acid side chain properties and high substitution frequencies in homologous proteins found in nature. In contrast, in certain embodiments, substitutions are non-conservative. A “non-conservative substitution” refers to the substitution of an amino acid in one class with an amino acid from another class.

The term “treating” (or “treat” or “treatment”) means slowing, interrupting, arresting, controlling, stopping, reducing, or reversing the progression or severity of a symptom, disorder, condition, or disease caused by a plant pathogen, and can include a total elimination of all disease-related symptoms, conditions, or disorders of affected plants.

The term “vector” as used herein refers to a DNA or RNA molecule capable of replication in a host cell and/or to which another DNA or RNA segment can be operatively linked so as to bring about replication of the attached segment. A plasmid is an exemplary vector.

This invention provides compositions and methods for controlling harmful pathogenic pests including fungal and oomycete pathogens.

Particularly, the invention relates to fungicidal and anti-oomycete proteins and variants thereof that have activity against Phytophthora infestans and Pseudoperonospora cubensis, and potentially for other damaging Phytophthora or other oomycete species, such as Phytophthora palmivora and Phytophthora ramorum. In some embodiments, the invention relates to fungicidal or anti-oomycete proteins having activity against Phytophthora infestans, Pseudoperonospora cubensis, Hyaloperonospora arabidopsidis, Phytophthora ramorum, Phytophthora sojae, Phytophthora capsici, Plasmopara viticola, Phytophthora cinnamomi, Phytophthora parasitica, Pythium ultimum, Albugo candida, Bremia lactucae, Phytophthora nicotianae, or any combination thereof.

In some non-limiting embodiments, the invention encompasses a nucleic acid molecule comprising a nucleotide sequence that encodes a protein that is toxic to or otherwise interferes with the pathogen, wherein the nucleotide sequence (a) encodes a protein comprising and amino acid sequence that has at least 70% to at least 99% sequence identity with any of RLTAQIRL, and RNTAQIPL or an effective fragment thereof; or (b) has at least 70% to 99% sequence identity to any of RLTAQIRL, and RNTAQIPL, or an effective fragment thereof; or (c) is a synthetic sequence of (a) or (b) that has codons optimized for expression in a transgenic organism.

The pesticidal peptides described herein may be applied to Solanum tuberosum. The application may be foliar, seed treatment, or on ground. Moreover, in other embodiments the pesticidal peptides of the present invention may be used to treat other species of the Solanum genus (e.g. S. tuberosum, S. lycopersicum, etc.). Food or crop plants that may be treated with the pesticidal peptides include soybean, wheat, maize, sugarcane, rice, tomato, grape, or potato. Crops may include, but are not limited to lettuces, ornamentals, peppers, eggplants, tomatoes, onion, citrus fruits, tobacco, cotton, potatoes, soy, legumes (e.g., alfalfa, clover, pea bean), or cucurbits (e.g., pumpkins, zucchini, cantaloupe, watermelon, cucumber).

The DNA construct used in certain embodiments of this method can further comprise a selectable marker gene. When the DNA construct further comprises a selectable marker gene, a transgenic plant of the invention is obtained by growing the plant, plant cell, or plant tissue under conditions requiring expression of the selectable marker gene for plant growth. This selectable marker gene can be selected from the group consisting of genes encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, and an aminoethylcysteine insensitive octopine synthase protein.

These methods of obtaining transgenic plants can also employ DNA constructs that further comprise a scoreable marker gene that functions in plants. In this case, expression of the scoreable marker gene is assayed to obtain a transgenic plant cell or a regenerated transgenic plant. Scoreable marker genes can be selected from the group consisting of genes encoding a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein, and a chloramphenicol acetyl transferase protein.

### EXAMPLES

Embodiments of this invention can be better understood by reference to the following examples. The foregoing and following description of embodiments of the invention and the various embodiments are not intended to limit the claims but are rather illustrative thereof. Therefore, it will be understood that the claims are not limited to the specific details of these examples. It will be appreciated by those skilled in the art that other embodiments of the invention may be practiced without departing from the spirit and the scope of the disclosure, the scope of which is defined by the appended claims.

### Example 1: Late Blight Detached Potato Leaf Assay

This protocol was used for performing detached potato leaf assays used Phytophthora infestans. The following materials were used: Petri Dishes (100×15 mm), Parafilm (PM-996), Heavy-duty paper towels (Uline, catalog number: S-13631BLU), cheesecloth, Spreader (Universal Medical, catalog number: HS86655), Sterile water, 70% ethanol, Fertilizer (Peters' Professional 20-20-20), Peat Lite special, P. infestans isolate US-23, Potato genotypes, obtained from seed savers. The following chemicals were used: MS medium, CaCO₃, V8 juice, agar.

Medium for growing P. infestans was made by soaking 50 g whole rye grains in 1100 ml water 24-72 hours. The grains were autoclaved for 30 minutes. The rye grain was strained out. 5% V8, 0.02% CaCO₃ and 16 gm agarose were added and then autoclaved for 30 minutes. Once the medium was cooled, 125 μg/ml carbenicillin disodium and 10 ug/ml rifamycin were added.

Potato plantlets were grown in MS medium for two weeks. The plantlets were kept in a growth chamber at 22° C. with 16/8 hr light/dark cycle. After two weeks the plantlets were transported into rockwool cubes. The rockwool cubes were kept in a growth chamber one week for acclimation. To maintain the health of plants, they were irrigated regularly.

P. infestans isolate US-23 was grown on a Rye-V8 media plate at 18° C. for 10-14 days. Three plugs (5 mm) were cut from the plate. They were in a new Rye-V8 media plate half centimeter apart. The plates were kept facing downward to avoid any moisture development on the plugs. Plates were be considered “usable” for assay when at least half of the plate was covered in mycelia.

P. infestans must periodically infect a living plant or leaves. Once every month the spores were used to infect leaves. Prepared simple agar plates (8 g agar per 1 liter of distilled water) were used.

Fresh healthy leaves from potato plant were sprayed with sterile water and touched the abaxial side of leaf with P. infestans plate surface. The leaf was placed on agar water plate and stored at room temperature for few days. Once the leaves showed infection, the zoospores were harvested by simply dissolving the leaves in 15 ml water and the spores were spread on V8 plates. Plates were kept in an 18° C. incubator. After 10 days, plugs were cut and placed on V8 plate to continue late blight cultures.

All solutions and centrifuge tubes with caps and correct labels, were kept on ice for the detached leaf assay. Uline paper towels before use (30 min at 15 psi) were autoclaved, and placed in the Bioassay boxes under a laminar hood.

Inoculum was prepared as follows. Sporangia were harvested from a 10-14-day old Rye-V8 cultured plate by flooding the plate with 5 ml ice-cold sterilized water (to expedite the release of zoospores). Plates were kept at 4° C. for 2 to 3 h to release zoospores and kept everything on ice throughout the procedure (plate and materials). Zoospores were harvested by filtering the liquid from each plate through two layers of cheesecloth (to remove mycelia). Motile zoospores were counted using a hemocytometer under a microscope. Concentration to 50,000 zoospores per ml was adjusted with water.

The fungicide solution was prepared by mixing 200 ul of Mancozeb fungicide with 400 ml of sterilized H2O.

The peptide treatments were as following. The peptide was prepared by dissolving it in water at a concentration of 2 mM. For example, if one tube contained 2 mg, and if the molecular weight of the peptide is 960 Da, then 1.04 ml water was added to create a 2 mM stock. Wherein the treatment was an inoculum plus the peptide: for each 10 μl drop; 2 μl peptide was mixed with 8 μl zoospores to give 400 μM peptide. Wherein the treatment was peptide plus water: as a control, 2 μl peptide was mixed with 8 μl water. To treat one trifoliate mix 24 μl of peptide and 96 μl of water were mixed. To make extra, 28 μl peptide plus 112 μl water was mixed. Wherein the treatment was inoculum alone plus water: as a control. 2 μl water was mixed with 8 μl zoospores. One tray of 3 trifoliate was treated with zoospores alone: 36 drops containing 8 μl of zoospores plus 2 μl of water was added each. Wherein the treatment was inoculum plus fungicide: as a control, 2 μl diluted commercial fungicide was mixed with 8 μl zoospores. To make the diluted fungicide, mix 1 ml of Mancozeb Flowable with Zinc from Bonide was mixed with 200 ml of water. Four 10 ul drops were used to infect each leaflet. Wherein the treatment was water alone: as a control, use 10 μl drops of autoclaved DI water was used.

Infection assay: Healthy, full-grown compound leaves having at least three leaflets from 5-8-week-old plants were collected. Each plate had at least three “trios” of compound leaves. Bioassay plate was lined with wet Uline heavy-duty paper towels. 30 mL water was added to each plate+towel to appropriately moisten the paper towel.

Symptom monitoring: The first symptoms were observed 3 to 5 days after inoculation as black/brown lesions, sporulation, and a water-soaked area at the point of pathogen inoculation. Pictures were taken each day of the progression of each plate, noting the plate and time/date in the image metadata. Symptoms were recorded when infection was large and cover the whole leaf in the case of highly susceptible genotypes after 5 to 7 days.

Results: The ability of NoPv1 peptide to block infection by P. infestans on detached potato leaves was set up as described above. Synthetic peptide was mixed with P. infestans at a concentration of 400 μM. After 6 days symptoms were clear. The NoPv1 peptide appears to show some protection compared to P.i. without peptide.

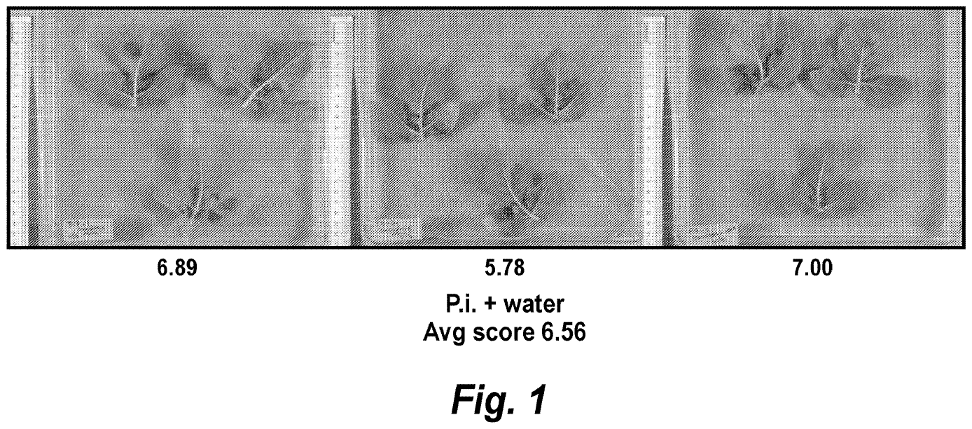

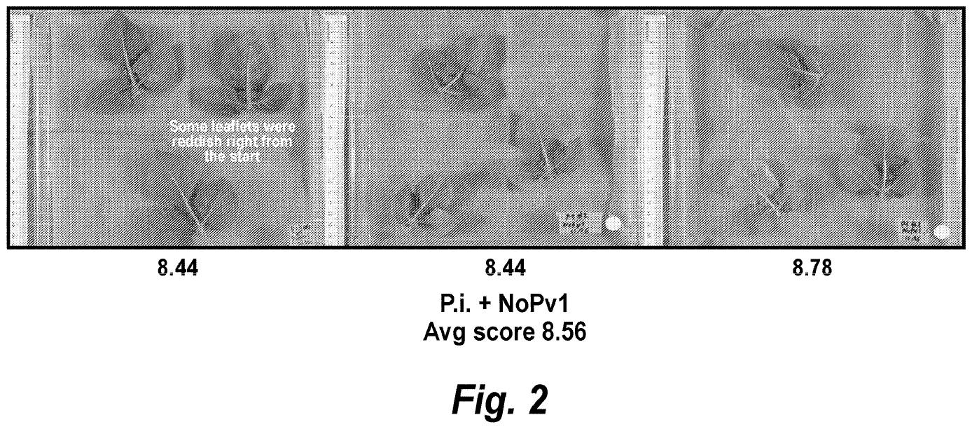

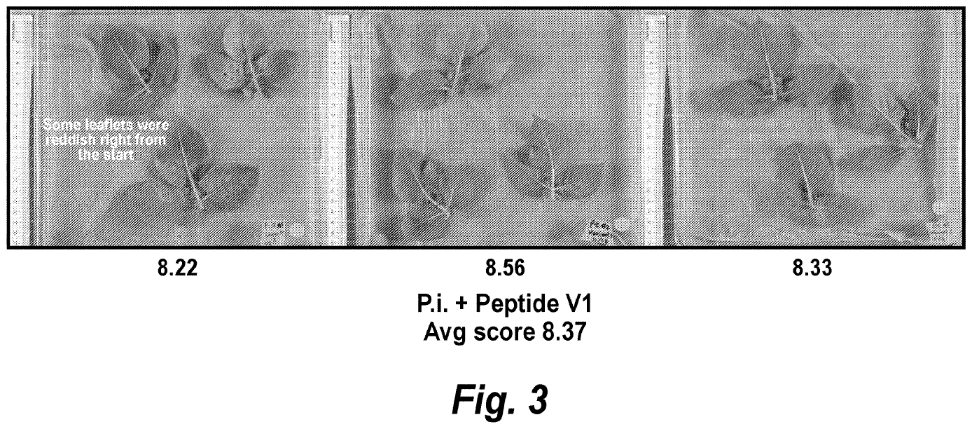

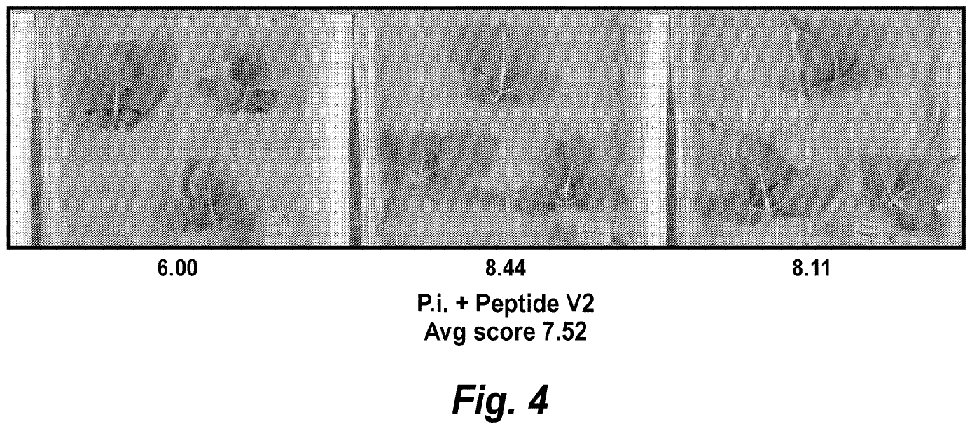

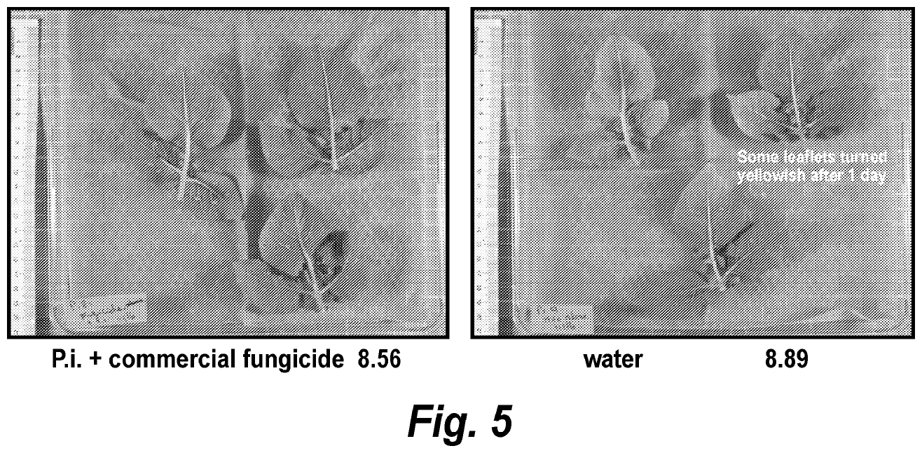

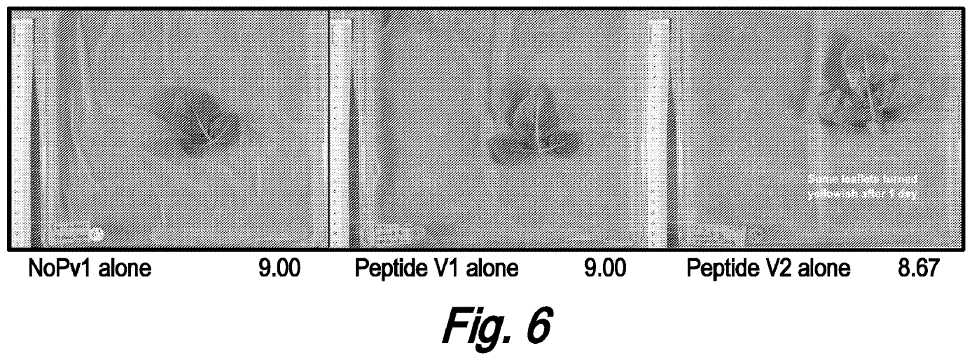

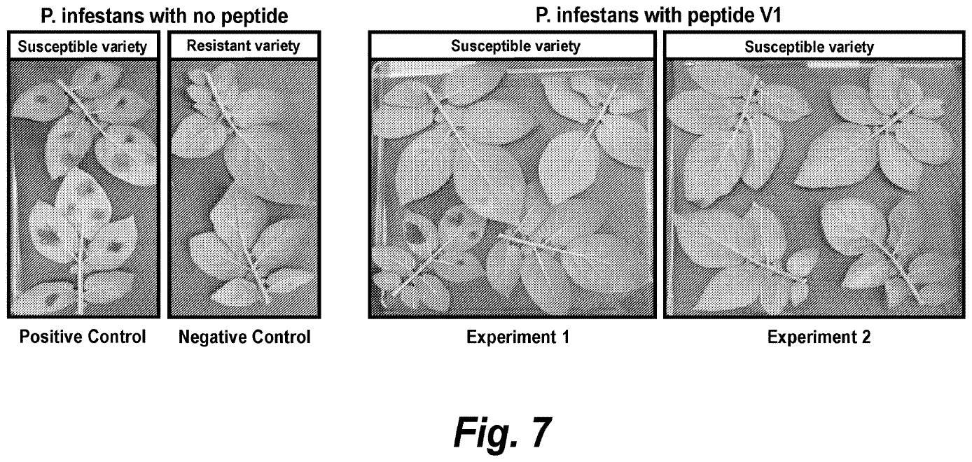

The experiment was repeated one week later, this time also testing the two variant peptides designed to bind more tightly to the target protein (cellulose synthase A2). The original NoPv1 peptide once again gave good protection. The first variant peptide (Peptide V1) was almost as good at reducing infection. It was harder to tell with the second variant peptide because the leaves started turning yellow shortly after they were removed from the plants, but it also appeared to give some protection. FIG. 1 is a photograph of the positive control of water 6 days after Phytophthora infection using 50 sporangia/ul final concentration. Each leaflet had 4 drops, 10 ul/drop and was kept on bench at 20 C. The average score (see below) for the positive control was 6.56. FIG. 2 is a photograph 6 days after Phytophthora infection and treatment with NoPV1 at 400 uM final concentration in the same conditions. An average score of 8.56 was attained. FIG. 3 is a photograph 6 days after Phytophthera infection and treatment with Peptide V1 at 400 uM final concentration in the same conditions. An average score of 8.37 was attained. FIG. 4 is a photograph 6 days after Phytophthora infection and treatment with Peptide V2 at 400 uM final concentration in the same conditions. An average score of 7.52 was attained. FIG. 5 is a photograph 6 days after Phytophthora infection and treatment with Bonide Mancozeb Flowable with Zinc diluted 1:1000. A score of 8.56 was attained. FIG. 6: is a photograph of the negative control of NoPV1, Peptide V1 and Peptide V2. Scores of 9.00 (NoPV1), 9.00 (Peptide V1), and 8.67 (variant 2) were attained. FIG. 7 is a photograph of Phytophthora infestans infection with a susceptible and resistant variety and with treatment with Peptide V1.

The experiment was repeated two more times. The first time some protection was observed with the first variant peptide (Peptide V1), but not with the original peptide or the second variant peptide. The second time, the first variant peptide (Peptide V1) gave excellent protection, and the second variant peptide and the original NoPv1 gave some protection.

Overall conclusion: the first variant peptide (Peptide V1) consistently works well. NoPv1 and the second variant are efficacious with more variability.

Symptom monitoring: The first symptoms were observed 3 days after inoculation as black/brown lesions, sporulation, and a water-soaked area at the point of pathogen inoculation.

Subsequently, symptoms enlarged and covered the whole leaf in the case of highly susceptible genotypes after 5 days. Leaves were visually assessed for the appearance of symptoms using a 1-9 scale (Karki et al., 2020) or quantified by using ImageJ software (Rueden et al., 2017), 5 days after inoculation.

FIGS. 1-7. The 1-9 scale was used to evaluate the late blight infection. The mean leaf area covered with blackish/brown lesions, sporulation, and water-soaking was calculated using ImageJ and assigned a value based on this area. 1=≥90%, 2=81-90%, 3=71-80%, 4=61-70%, 5=41-60%, 6=21-40%, 7=10-20% with cell death at the point of inoculation, 8=≤10% with cell death at the point of inoculation, and 9=0% infection, no visible symptoms, clean leaves.

### Example 3: Production of Thioredoxin-Peptide V1 Fusion Protein

2 mL of TB+glucose medium containing 2 μL of carbenicillin (100 mg/mL) was inoculated in a culture tube with the BL21 E. coli glycerol stock and incubate overnight at 37° C. with shaking (250 rpm). 100 mL of TB+glucose medium containing 100 μL of carbenicillin (100 mg/mL) was inoculated in a 500 ml baffle flask with 750 μL of the overnight culture.

The culture was grown at 37° C. with shaking for approximately 1.5 hours until reaching an optical density at 600 nm (OD600) of 0.6. Isopropyl β-D-1-thiogalactopyranoside (IPTG) was added to a final concentration of 0.5 mM to induce protein expression. The culture was incubated overnight at 18° C. with shaking.

[Cells were pelleted by centrifugation at 5,000×g for 20 minutes at 4° C. The supernatant was discarded, and the pellet stored at −80° C. if not proceeding immediately to purification. To lyse the cells, the cell pellet was resuspended in 10 mL of lysis buffer (20 mM sodium phosphate, 300 mM NaCl, 10 mM imidazole, pH 7.4) with protease inhibitors. Cells were lysed by sonication at 70% power for 30 seconds (repeat 4 times with 1-minute intervals). The sonicated sample was centrifuged at 16,000×g for 20 min. The supernatant was collected in a falcon tube.

Protein purification was accomplished using nickel columns. The columns were packed with the appropriate amount of Ni-NTA resin (2 mL). The storage buffer was allowed to drain from the resin. The sample was prepared by mixing the protein extract with an equal volume of Equilibration buffer (10 mM sodium phosphate, 300 mM NaCl, 10 mM imidazole, pH 7.4). The column was equilibrated with two resin-bed volumes (2 mL) of Equilibration buffer. Allow the buffer to drain from the column. 5 mL of prepared protein was loaded onto the equilibrated column and mix on a rotator for 30 min. The flowthrough was drained from the column. The column was washed with two bed-resin volumes (2 mL) of the wash buffer (20 mM sodium phosphate, 300 mM NaCl, 25 mM imidazole, pH 7.4) to remove nonspecifically bound proteins. The target protein was eluted with two resin-bed volumes of elution buffer (20 mM sodium phosphate, 300 mM NaCl, 250 mM imidazole, pH 7.4). Purified fractions were analyzed by SDS-PAGE to confirm purity.

Protein from a 100 mL culture of thioredoxin fusion protein produced 6 mL of eluted protein at 2.0 mg/mL, resulting in a peptide concentration of 117.6 PM. This protein needed to be concentrated approximately 7 times. The protein sample was added to the centrifugal concentrator (VIVASPIN) reservoir (5,000 MW cutoff). The sample was centrifuged in the microcentrifuge (Thermofisher, Legend Micro 17R Microcentrifuge 24×1.5/2.0 mL Rotor) at an appropriate speed (around 7000×g) for a sufficient time to concentrate the protein 10 fold. Exchange buffer was added (Tris 5 mM, NaCl 5 mM) up to the initial protein volume and centrifuge to obtain the initial volume. This process was repeated three times to dilute the elution buffer 1000 fold. If needed, the exchange buffer was added to obtain the required protein concentration. Finally, 300 μg of protein was combined with 1.0 μL of Enterokinase (16,000 U/mL) and incubated at 25° C. overnight.

### Example 4: Broad Spectrum Pathogen Testing of Peptide V1

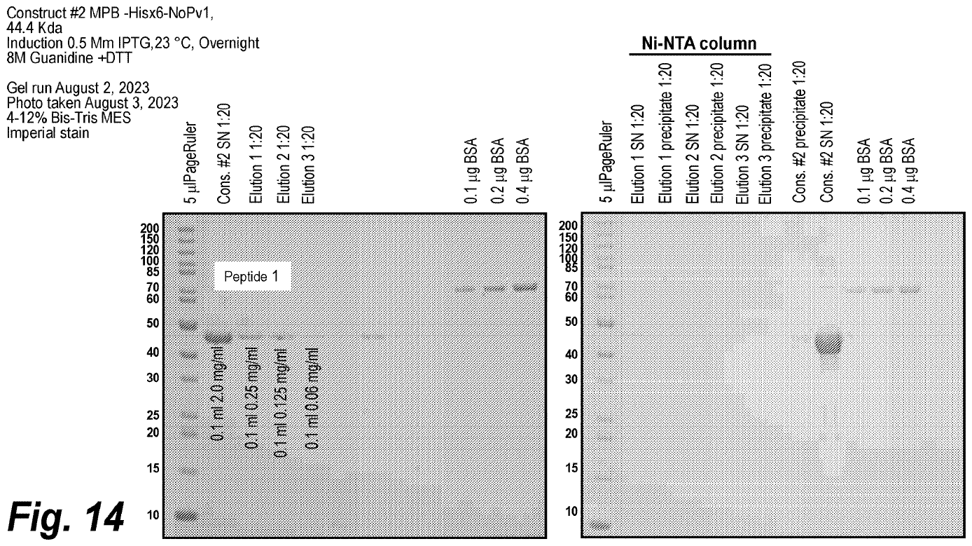

The experiment consists of a completely randomized block design with four replications. Two treatments (Orondis Opti and Peptide V1 LI700) and two controls non-treated (non-treated inoculated and non-treated non-inoculated) were tested. Experimental plots were two leaves on one square foot plastic humidity chamber containing three layers of sterile paper towels. Cucumber leaves (3rd to 4th leaf) were harvested on Oct. 31, 2023. Treatments were applied on Oct. 31, 2023 as a foliar spray application and treatments were inoculated by spraying with Pseudoperonospora cubensis suspension of 106 spores/ml on Nov. 1, 2023. The non-treated non-inoculated control was sprayed with water. Humidity chambers were incubated at 21° C. for 12 hr with light and 19° C. with 12 hr of darkness. A visual disease rating scale was used to measure the disease severity (FIG. 14: A, Treatment 1—Orondis Opti; B, Treatment 2—Peptide V1+Adjuvant LI700; C, Treatment 3—Non-treated, Inoculated; and D, Treatment 4—Non-treated, Non-inoculated). Disease severity percentage (%) per leaf was assessed every 3 days post-inoculation until November 13. One-way analysis of variance (ANOVA) was fitted to compare the mean effects of each treatment on the final day of disease severity and Area Under the Disease Progress Curve values (AUDPC) using ARM (Gylling Data Management, Brookings, SD). Fisher's protected least significant differences (LSD) test was applied to separate means.

Both disease severity on the last day of the experiment and AUDPC were significantly lower in inoculated plant leaves treated with Orondis Opti and Peptide V1+Adjuvant LI700 than in the inoculated but non-treated control (Table 1). Orondis Opti was not statistically different from the non-treated non-inoculated control. Between the two treatments, both disease severity and AUDPC were significantly lower with Orondis Opti than with Peptide V1+Adjuvant L1700. No phytotoxicity was observed in the experiment.

The results are shown in Table 1 below:

| | | Disease | | | |
|---|---|---|---|---|---|
| Treatments | Rate | Severity²(%) | | AUDPC^{y} | |
| Non-treated, | — | 0.00 | c^{x} | 0.00 | c |
| Non-inoculated | | | | | |
| Non-treated, | — | 68.50 | a | 320.25 | a |
| Inoculated | | | | | |
| Orondis Opti | 32.0 fl oz | 0.00 | c | 0.00 | c |
| Peptide V1 | 400 μMol | 34.50 | b | 115.875 | b |
| LI700 | 0.25 v/v | | | | |

Assays of P. capsici on pepper plants were done with three treatments: a water control, growers' standard fungicide, and 400 uM Peptide V1. For each treatment 10 plants were used. Soil was drenched with 50 ml of treatment per plant. Required 0.2 g peptide. Treatments were applied, wait 1 day, then inoculated with pathogen.

Plants were exposed to Phytophthora erythroseptica (pink rot). An inoculum was prepared by the following. An agar plug (5 mm in diameter) was taken from the margin of a 3-day-old culture of P. erythroseptica was placed in 15 mL clarified V8 broth in a 50 mL plastic disposable sterile centrifuge tube (Fisher Scientific). Tubes were placed in the dark at 18° C. for 3 days to allow growth of the pathogen. The V8 broth was then decanted and mycelium washed twice with 10 mL sterile distilled water. Following the final wash, cultures were immersed in 10 mL sterile lake water (obtained locally) and kept at 18° C. under continuous fluorescent light. After incubation for 24 h, lake water was replaced with 15 mL fresh sterile lake water, and cultures were incubated for a further 24 h at 18° C. under continuous fluorescent light. Following incubation, cultures were chilled at 9° C. for 1.5 h, then placed in the dark at room temperature (22° C.) for 30 min to encourage zoospore release.

Tubers (cultivar Snowden) were washed in cold water and allowed to air-dry in preparation for inoculation. One healthy looking apical eye of each tuber was circled using a marker, then inoculated with a 20 mL droplet of inoculum containing 2×10\hat{ }4 zoospores/mL (as determined with a haemocytometer). Controls were inoculated with sterile pond water only. The inoculum droplet was undisturbed for 5 min, after which tubers were wrapped in wet paper towel and placed in a sealed plastic tub lined with wet paper towels. Tubers were incubated for 14 days at 15-18° C. and 100% RH. Inoculated tubers were sectioned longitudinally from the point of inoculation and covered with a wet paper towel for 30 min to allow the development of pink coloration in internal infected tissues.

The procedure for the pink rot is based on this paper: https://bsppjoumals.onlinelibrary.wiley.con/doi/pdf/10.1046/j.1365-3059.2001.00566.x (also attached). Peters, RD, Sturz, AV, Matheson, BG, Aresnault, W J, and Malone, A. Metalaxyl sensitivity of isolates of Phytophthora erythroseptica in Prince Edward Island. (2001) Plant Pathology. 50: 302-309

### Example 5: Functional Stability of the Peptide

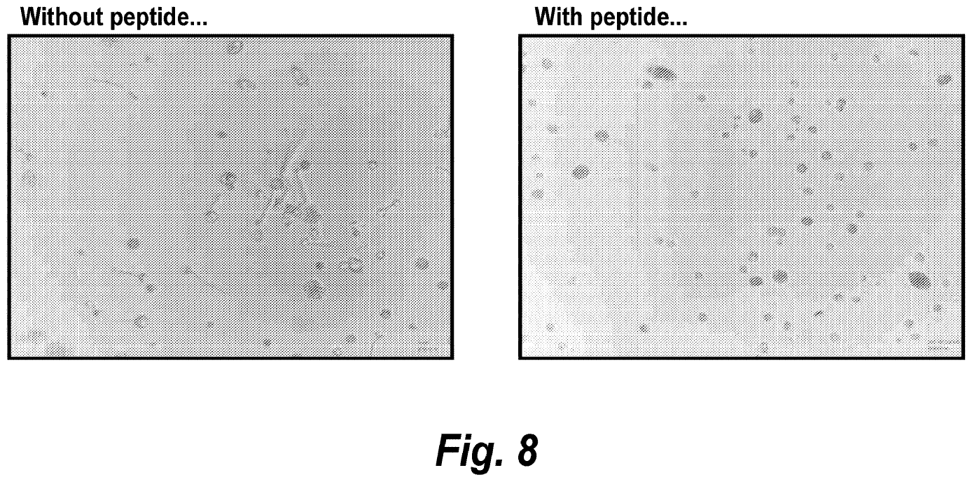

Under laboratory conditions, Peptide V1 adhered to the leaves, preventing it from being washed away by rain. It remained active even after exposure to temperatures of 40° C. for 9 days and direct sunlight. To assess its stability under direct sunlight, dried droplets of the peptide solution were placed on Petri dishes, which were then placed outdoors in direct sunlight for up to 16 days. At various time points, zoospores were added to re-dissolve the dried peptide and test its activity in a germination assay. After 8 days (equivalent to 48 cumulative hours of direct sunlight exposure), we observed a slight amount of germination, as seen in FIG. 8.

### Example 6: Physical Stability

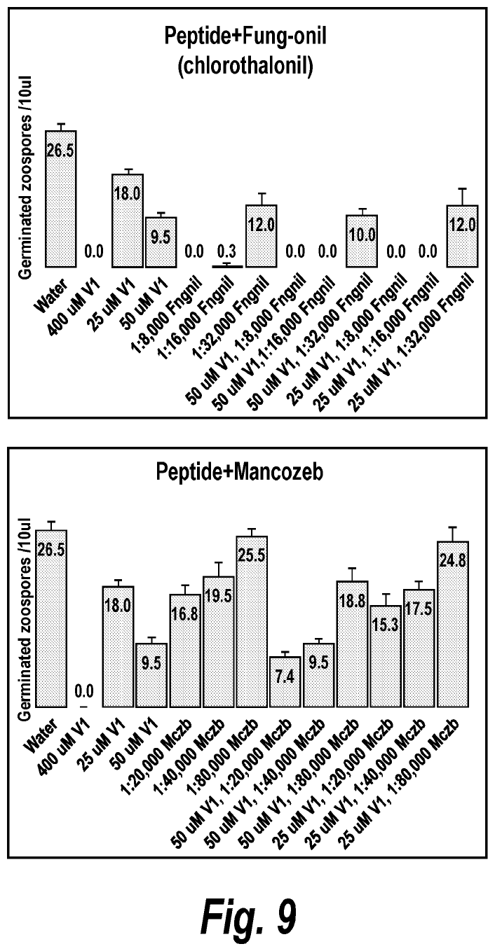

Plants were sprayed with Peptide V1 without the use of a spreader and were placed outdoors, thus being exposed to environmental conditions. Leaf samples were collected at intervals of 0, 1, 2, 4, 8, and 16 days and were subsequently sent to ISU for peptide quantitation using mass spectrometry. The peptide's concentration on the leaves remained relatively stable over the course of 16 days in the presence of Triton, as seen in FIG. 9. However, some control samples with Triton alone showed sporadic peptide cross contamination, resulting in notably large error bars.

### Example 7: Finding a Synergistic or Additive Effect with Chemical Fungicides

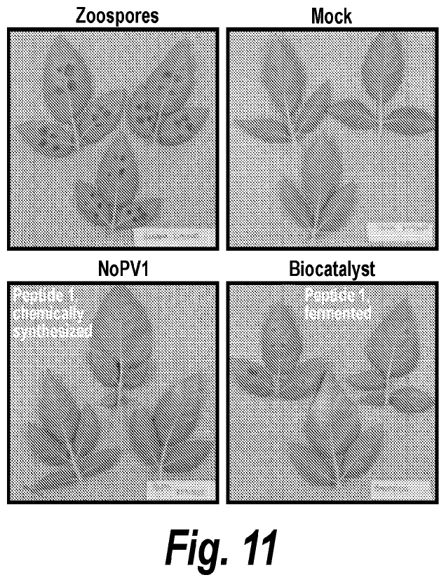

Results from the germination assays did not indicate any evidence of additive or synergistic effects between Peptide V1 and either Mancozeb or Fungonil (Chlorothalonil). Similarly, the severity of symptoms observed in the plant assays were approximately the same across the non-treated control group, plants treated with a low dose of Peptide V1 (25 μM), those treated with a low dose of Mancozeb, and those treated with the combination of both as seen in FIG. 11.

### Example 8: Identifying a Spreader and a Formulation to Mix with Peptide V1

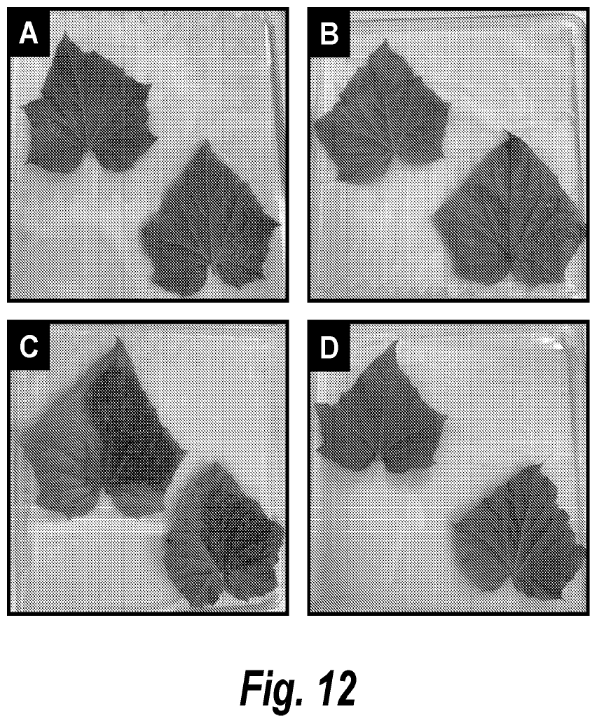

Whole plant assays demonstrated that the combination of the peptide and LI-700 reduced disease severity compared to using Peptide V1 alone. In the whole plant assays with a 400 μM peptide concentration, disease reduction was approximately 60% when applying the peptide alone and increased to 70% when applying it in combination with LI-700 as seen in FIG. 12.

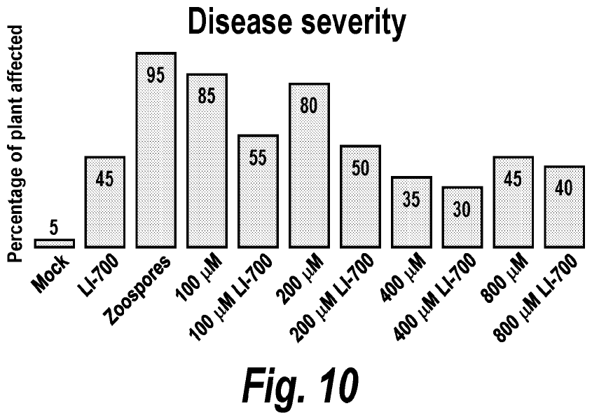

An experiment conducted with large plants, where various Peptide V1 concentrations were evaluated with and without LI-700, revealed that the best protection was achieved with a combination of 400 μM peptide and 400 μM peptide plus LI-700 as seen in FIG. 10.

### Example 9: Making Peptide V1 in E. coli

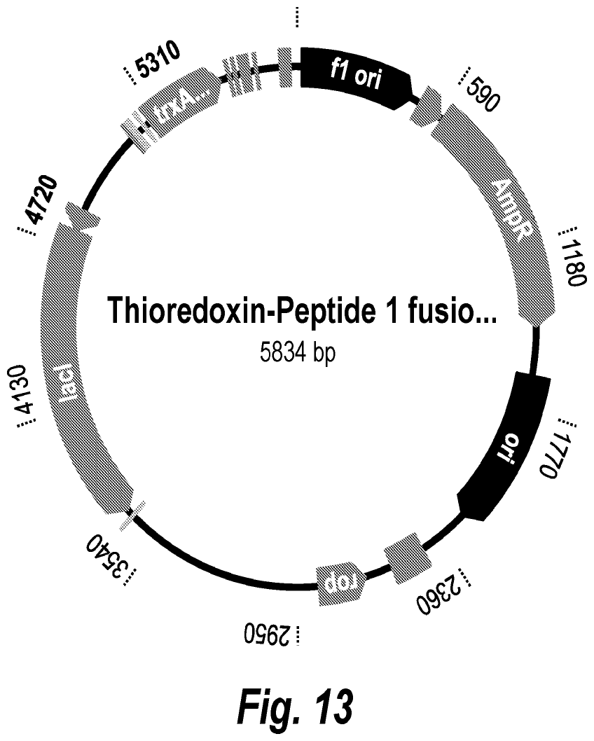

Peptide V1 was expressed in E. coli with the plasmid shown in FIG. 13. In the zoospore germination assay, this peptide completely inhibited zoospore germination. In the detached leaf assay, while it allowed the development of minor late blight lesions, it is noteworthy that these lesions did not progress, as shown in FIGS. 11 & 12.

Further, three other fusion constructs were designed for expressing Peptide V1 in E. coli. One of these constructs, comprising a 6-His tag, maltose-binding protein, a protease cleavage site, and the peptide (MW~46 kDa), showed robust expression but formed inclusion bodies. These inclusion bodies were successfully solubilized using urea or guanidine, followed by purification on nickel columns. Notably, the protein remained soluble after removing the denaturant. In guanidine purification, only about a quarter of the protein was recovered, and given its initial purity, column purification may not be necessary.

A construct that gave good expression featured a 6×His tag, maltose binding protein, a linker, and a protease cleavage site (enterokinase) within the pET28b vector. Growth conditions used TB-glucose media, induced with 0.5 mM IPTG, and subsequent overnight growth at 20 degrees Celsius. This resulted in an approximate yield of 5 grams per liter of culture, with the peptide accounting for around 2% of the fusion protein's mass, equivalent to 0.1 grams per liter of culture, but with the protein within inclusion bodies as seen in FIG. 14.

To release the protein from inclusion bodies, large cultures (100 ml) were induced, and inclusion bodies were solubilized using either Guanidine-DTT or Urea-DTT. However, post-dialysis, the protein precipitated. To address this issue, the minimum concentration of either urea or guanidine necessary to dilute the inclusion bodies was 6M in both cases. Furthermore, additives were identified that aided in refolding the protein and maintaining its solubility. In the case of urea-solubilized protein, the most effective additive for maintaining solubility was deoxycholic acid, followed by CHAPS, Triton X-100, and Tween-20.

### Example 10: Peptide V1 Mode of Action

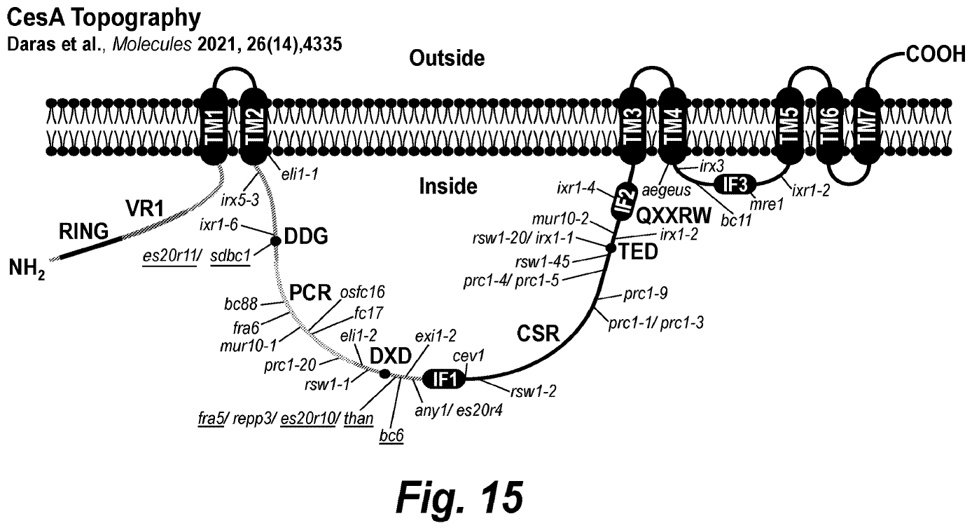

NoPv1 was originally identified using yeast 2-hybrid binding to the soluble cytoplasmic portion of Plasmopara viticola CesA2 (Colombo, M., Masiero, S., Rosa, S. et al. NoPv1: a synthetic antimicrobial peptide aptamer targeting the causal agents of grapevine downy mildew and potato late blight. Sci Rep 10, 17574 (2020). https://doi.org/10.1038/s41598-020-73027-x). NoPv1 Variant 1 (Peptide V1) must be getting inside Phytophthora zoospores in order to inhibit cellulose synthesis. The presence of positive charges in a peptide enhances its ability to penetrate cells. Peptide V1 contains 3 positive charges, including the N-terminus, within just 8 amino acids as depicted in FIG. 15.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary of the present invention and not limiting thereof. The scope of the invention is therefore set out in the appended claims.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

1. A polypeptide comprising an amino acid sequence having at least 70% identity to RLTAQIRL, or RNTAQIPL, or a combination thereof.

2. The polypeptide of claim 1, wherein an agriculturally effective amount is applied to a crop.

3. The polypeptide of claim 2, wherein the crop is potato, soy, maize, tomato, pepper, rice, grape, cucurbits, lettuce.

4. The polypeptide of claim 1, wherein an agriculturally effective amount prevents, inhibits, or deters infection of a crop with an oomycete.

5. An anti-oomycete composition comprising a polypeptide having an amino acid sequence having at least 90% identity to RLTAQIRL, or RNTAQIPL, or a combination thereof, and further comprising an agrochemical carrier, adjuvant, stabilizer or a second agrochemical active agent.

6. The anti-oomycete composition of claim 5, wherein the anti-oomycete composition is effective against infection or ameliorates damage to a plant by an oomycete.

7. The anti-oomycete composition of claim 5, wherein the oomycete is Phytophthora infestans, Hyaloperonospora arabidopsidis, Phytophthora ramorum, Phytophthora sojae, Phytophthora capsici, Plasmopara viticola, Phytophthora cinnamomi, Phytophthora parasitica, Pythium ultimum, Albugo candida, Bremia lactucae, Phytophthora nicotianae, Pseudoperonospora cubensis, or Brenda lactucae, or any combination thereof.

8. The anti-oomycete composition of claim 6, wherein the plant is a crop.

9. The anti-oomycete composition of claim 8, wherein the crop soybean, wheat, maize, sugarcane, rice, tomato, grape, or potato, lettuces, ornamentals, peppers, eggplant, onion, citrus fruits, tobacco, cotton, legumes, or cucurbits.

10. A bacterium expressing a peptide having 80% identity to RLTAQIRL, or RNTAQIPL.


SEQUENCE LISTING (3 sequences)

SEQ ID NO: 1 - protein, 8 aa, unidentified
RLTAQCRL

SEQ ID NO: 2 - protein, 8 aa, unidentified
RLTAQIRL

SEQ ID NO: 3 - protein, 8 aa, unidentified
RNTAQIPL